(19) United States
(12) Patent Application Publication
(10) Pub. No.: US 2009/0254018 A1
(43) Pub. Date: Oct. 8, 2009

(54) Electrode assembly for freezing-type iontophoresis device

(76) Inventors: Mizuo Nakayama, Tokyo (JP); Takehiko Matsumura, Tokyo (JP); Hidero Akiyama, Tokyo (JP); Akihiko Matsumura, Tokyo (JP)

Correspondence Address: SEED INTELLECTUAL PROPERTY LAW GROUP PLLC, 701 FIFTH AVE, SUITE 5400, SEATTLE, WA, 98104, US

(21) Appl. No.: 11/990,882

(22) PCT Filed: Aug. 24, 2006
(86) PCT No.: PCT/JP2006/316625

§ 371 Date: May 6, 2009

(30) Foreign Application Priority Data
Aug. 24, 2005 (JP) 2005-243008

(51) Int. Cl. A61N 1/30

(52) U.S. Cl. 604/20

(57) ABSTRACT

An iontophoresis device capable of stably holding a drug during a storage period and of transdermally administering the drug to an organism at a high transport number during use. The iontophoresis device includes an electric power source. A first electrode assembly may be electrically coupled to the electric power source to store and transdermally administer an ionic drug to an organism via iontophoresis. The first electrode assembly stores the ionic drug at a temperature less than or equal to zero degrees Celsius and administers the ionic drug at a temperature greater than zero degrees Celsius. A second electrode assembly may be electrically coupled to the electric power source as a counter electrode to the first electrode assembly.

### BACKGROUND

1. Technical Field

The present disclosure relates to a technique of transdermally administering various ionic drugs (transdermal drug delivery) by iontophoresis. In particular, the present disclosure relates to a composition and an electrode assembly for iontophoresis each of which is useful in holding a drug stable for a substantially long time period and in transdermally administering the drug at a high transport number during use.

2. Description of the Related Art

A method of introducing (permeating) an ionic drug placed on the surface of the skin or mucosa (hereinafter, merely referred to as “skin”) of a predetermined site of an organism into the body through the skin by giving the skin an electromotive force sufficient to drive the ionic drug is called iontophoresis (iontophorese, ion introduction method, ion permeation therapy) (e.g., JP 63-35266 A).

For example, positively charged ions are driven (transported) into the skin on the side of an anode (positive electrode) in an electric system of an iontophoresis device. On the other hand, negatively charged ions are driven (transported) into the skin on the side of a cathode (negative electrode) in the electric system of the iontophoresis device.

Conventionally, a large number of such iontophoresis devices as described above have been proposed (See e.g., JP 63-35266 A, JP 04-297277 A, JP 2000-229128 A, JP 2000-229129 A, JP 2000-237327 A, JP 2000-237328 A and WO 03/037425 A1).

In the conventional iontophoresis devices described above, the drug stored prior to delivery may degrade due to, for example, leakage and decomposition of the drug depending on, for example, the length of the storage period and the type of the drug. Therefore, it is desirable for an iontophoresis device to stably hold a drug in order to prevent the degradation of the drug.

### BRIEF SUMMARY

In some embodiments an iontophoresis device may be capable of holding a drug stable during a storage period and of transferring the drug into an organism at a high transport number during use. Administering the drug to the organism at the high transport number during use may secure a sufficient therapeutic effect.

According to one embodiment an iontophoresis device includes an electric power source, a first electrode assembly electrically coupled to the electric power source to store and transdermally administer an ionic drug to an organism via iontophoresis wherein the first electrode assembly stores the ionic drug at a temperature less than or equal to zero degrees Celsius and administers the ionic drug at a temperature greater than zero degrees Celsius, and a second electrode assembly electrically coupled to the electric power source as a counter electrode to the first electrode assembly.

According to one embodiment an electrode assembly for iontophoresis includes a first electrode electrically coupled to the electric power source to have a same polarity as a component of the ionic drug, a first electrolyte solution holding portion impregnated with a first electrolyte solution, the first electrolyte solution holding portion disposed adjacent to the first electrode, a first ion exchange membrane that substantially passes ions having a polarity that is the same as a polarity of the ionic drug and that substantially blocks ions having a polarity that is opposite the polarity of the ionic drug, the ion exchange membrane disposed adjacent to the first electrolyte solution holding portion, a drug holding portion impregnated with the ionic drug, the drug holding portion disposed adjacent to the first ion exchange membrane, and a second ion exchange membrane that substantially passes ions having a polarity opposite the polarity of the ionic drug and that substantially blocks ions having a polarity that is the same as a polarity of the ionic drug, the second ion exchange membrane disposed adjacent to the drug holding portion wherein the ionic drug is transdermally administered to the organism via the second ion exchange membrane with a substantially high transport number.

As described above, the electrode assembly for iontophoresisis frozen. Therefore, an ionic drug can be stably held during a storage period, and the high transport number of the ionic drug can be secured at the time of use. The leakage of an ionic drug from the drug solution holding portion to another member and the decomposition of the ionic drug can be prevented because the electrode assembly for iontophoresis according to at least one embodiment is frozen. In addition, even when the electrode assembly is stored for a long time period, there is no need to add a preservative or the like that may inhibit the transport number of an ionic drug. Therefore, the high transport number of the ionic drug can be secured at the time of use.

### BRIEF DESCRIPTION OF THE SEVERAL VIEWS OF THE DRAWING(S)

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a schematic illustration of an electrode assembly for iontophoresis, according to one illustrated embodiment.

FIG. 2 is an iontophoresis device including the electrode assembly for iontophoresis, according to one illustrated embodiment.

### DETAILED DESCRIPTION

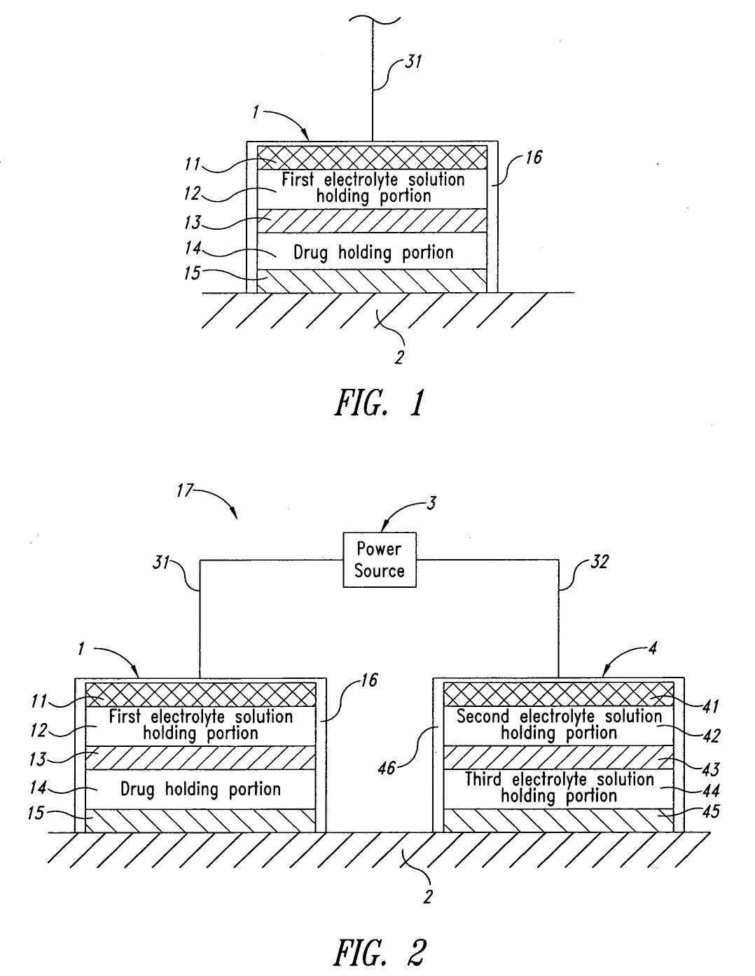

FIG. 1 shows an electrode assembly 1 for iontophoresis, according to one embodiment, placed on an organism 2 (e.g., skin). The electrode assembly 1 for iontophoresis may include a first electrode 11 electrically coupled to an electric power source 3 to have a same polarity as a drug component of an ionic drug. The first electrode 11 may, for example, be coupled to the electric power source 3 via a cord 31. A first electrolyte solution holding portion 12 may be impregnated with a first electrolyte solution and may be placed adjacent to the first electrode 11. A first ion exchange membrane 13 may substantially pass ions having a polarity that is the same as a polarity of the ionic drug and substantially block ions having a polarity that is opposite the polarity of the ionic drug. The first ion exchange membrane 13 may be placed adjacent to the first electrolyte solution holding portion 12. A drug solution holding portion 14 may be impregnated with the ionic drug and placed adjacent to the first ion exchange membrane 13. A second ion exchange membrane 15 may substantially pass ions having a polarity opposite the polarity of the ionic drug and substantially block ions having a polarity that is the same as a polarity of the ionic drug. The second ion exchange membrane 15 may be placed adjacent to the drug solution holding portion 14. The electrode assembly 1 may be housed in an enclosure 16 (e.g., cover or container) and may be frozen.

After the electrode assembly 1 is defrosted, the electrode assembly 1 may be used as a working electrode assembly for transdermally administering an ionic drug in an iontophoresis device. FIG. 2 shows an iontophoresis device 17 including the defrosted electrode assembly 1 used for iontophoresis, the electric power source 3, and a non-working electrode assembly 4 as a counter electrode of the electrode assembly 1 placed on the organism 2 (e.g., skin).

The electrode assembly 1 of FIG. 2 may have a same configuration as that shown in FIG. 1 except that it is defrosted. The electrode assembly 1 may be electrically coupled to the electric power source 3 on a side having the same polarity as that of the ionic drug. The electrode assembly may, for example, be electrically coupled to the electric power source 3 via a cord 31.

The non-working electrode assembly 4 may include a second electrode 41 electrically coupled to the electric power source 3 to have a polarity opposite that of the first electrode 11. The second electrode 41 may, for example, be electrically coupled to the electric power source 3 via a cord 32. A second electrolyte solution holding portion 42 may be impregnated with a second electrolyte solution and may be placed adjacent to the second electrode 41. A third ion exchange membrane 43 may substantially pass ions having a polarity opposite the polarity of the ionic drug and substantially block ions having a polarity that is the same as a polarity of the ionic drug. The third ion exchange membrane 43 may be placed adjacent to the second electrolyte solution holding portion 42. A third electrolyte solution holding portion 44 may be impregnated with a third electrolyte solution and may be placed adjacent to the third ion exchange membrane 43. A fourth ion exchange membrane 45 may substantially pass ions having a polarity that is the same as a polarity of the ionic drug and substantially block ions having a polarity that is opposite the polarity of the ionic drug. The fourth ion exchange membrane 45 may be placed adjacent to the third electrolyte solution holding portion 44. The non-working electrode assembly 4 may be housed in an enclosure 46 (e.g., a cover or container). The non-working electrode assembly 4, as described above, is exemplified as one embodiment. Therefore, in the iontophoresis device 17 including the first electrode assembly 1 for iontophoresis, the non-working electrode assembly 4 is not limited to the above embodiment.

Upon energization of the iontophoresis device 17 by means of the electric power source 3, the ionic drug migrates by virtue of an electric field, and may be transdermally administered to the organism 2 via the second ion exchange membrane 15. In this case, an ion having a polarity opposite that of the ionic drug is prevented from transferring from the side of the organism 2 to the side of the drug solution holding portion 14 by the action of the first and second ion exchange membranes 13 and 15. In addition, the movement of H+or OH− generated at the first electrode 11 to the side of the organism 2 (e.g., skin) is suppressed. As a result, the ionic drug can be administered efficiently and stably for a long time period while a change in pH on a surface of the organism 2 (e.g. skin 2) is suppressed.

The temperature at which the electrode assembly 1 may be frozen can be appropriately selected in accordance with, for example, the type and stability of the ionic drug. The temperature selected to freeze the electrode assembly 1 may, for example, be between 0 to−80° C.

The electrode assembly 1 may be frozen by means of a conventionally known freezer adjusted to such temperature at which the electrode assembly 1 is frozen as described above. In addition, the electrode assembly 1 may, for example, be defrosted using a conventionally known defrosting device or by leaving the electrode assembly 1 in a room at a temperature which the electrode assembly 1 may be defrosted. The defrosting temperature may be selected in accordance with, for example, the type and stability of the ionic drug. During and after the defrosting, extra water adhering to the outside of the electrode assembly 1 may be appropriately removed by means of a conventionally known drying device before the electrode assembly 1 is used.

In addition, an inactive electrode made of a conductive material such as, for example, carbon or platinum may be used to serve as the electrode assembly 1 for iontophoresis.

The first electrolyte solution holding portion 1 may include a thin film that has the property of holding an electrolyte solution by being impregnated with the electrolyte solution. The thin film may be made of the same material as that used for a drug solution holding portion to be described later.

A desired one can be appropriately used as the electrolyte solution depending upon the conditions such as a drug to be applied. However, an electrolyte solution that damages the skin of an organism owing to an electrode reaction should be avoided. An organic acid or a salt thereof present in a metabolic cycle of an organism is preferable as the electrolyte solution in the present invention in terms of harmlessness. For example, lactic acid and fumaric acid are preferable. Specifically, an aqueous solution of 1 M of lactic acid and 1 M of sodium fumarate (1:1) is preferable.

A cation exchange membrane and an anion exchange membrane are preferably used together as ion exchange membranes to be used for an electrode assembly. Preferable examples of the cation exchange membrane include NEOSEPTAs (CM-1, CM-2, CMX, CMS, CMB, and CLE04-2) manufactured by Tokuyama Co., Ltd. Preferable examples of the anion exchange membrane include NEOSEPTAs (AM-1, AM-3, AMX, AHA, ACH, ACS, ALE04-2, and AIP-21) manufactured by Tokuyama Co., Ltd. Other preferable examples include: a cation exchange membrane that includes a porous film having cavities a part or whole of which are filled with an ion exchange resin having a cation exchange function; and an anion exchange membrane that includes a porous film having cavities a part or whole of which are filled with an ion exchange resin having an anion exchange function.

The above-mentioned ion exchange resins can be fluorine-based, each including a perfluorocarbon skeleton having an ion exchange group, and hydrocarbon-based, each including a nonfluorinated resin as a skeleton. From the viewpoint of convenience of production process, hydrocarbon-based ion exchange resins may be used. The filling rate of the porous film with the ion exchange resin, which varies depending on the porosity of the porous film, can be, for example, 5% to 95% mass, 10% to 90% mass, or 20% to 60% mass.

The ion exchange group in the above-mentioned ion exchange resin is not particularly limited in so far as it is a functional group that generates a group having negative or positive charge in aqueous solutions. Such functional group may be present in the form of a free acid or a salt. Examples of a cation exchange group include a sulfonic group, a carboxylic acid group, and a phosphonic acid group. Of those, a sulfonic group may be preferable. Examples of a counter cation for the cation exchange group include: alkali cations such as a sodium ion and a potassium ion; and ammonium ions. Examples of an anion exchange group may include a primary amino group, a secondary amino group, a tertiary amino group, a quaternary amino group, a pyridyl group, an imidazole group, a quaternary pyridium group, and a quaternary imidazolium group. Of those, a quaternary ammonium group or a quaternary pyridium group may be preferable. Examples of a counter cation for the anion exchange group may include halogen ions such as, for example, chlorine ions and hydroxy ions.

The above-mentioned porous film is not particularly limited and any porous film can be used that may be in the form of a film or sheet that has a large number of pores communicating both sides thereof. To satisfy both high strength and flexibility, the porous film may be made of a thermoplastic resin. Examples of the thermoplastic resin comprising the porous film may include polyolefin resins such as, for example, homopolymers or copolymers of α-olefins such as ethylene, propylene, 1-butene, 1-pentene, 1-hexene, 3-methyl-1-butene, 4-methyl-1-pentene, and 5-methyl-1-heptene; vinyl chloride-based resins such as polyvinyl chloride, vinyl chloride-vinyl acetate copolymers, vinyl chloride-vinylidene chloride copolymers, and vinyl chloride-olefin copolymers; fluorine-based resins such as polytetrafluoroethylene, polychlorotrifluoroethylene, polyvinylidene fluoride, tetrafluoroethylene-hexafluoropropylene copolymers, tetrafluoroethylene-perfluoroalkyl vinylether copolymers, and tetrafluoroethylene-ethylene copolymers; polyamide resins such as nylon; and polyimide resins. Of those, polyolefin resins may be preferable when considering, for example, mechanical strength, flexibility, chemical stability, and chemical resistance. Of those, polyethylene or polypropylene may be preferable.

The properties of the porous film made of the above-mentioned thermoplastic resin are not particularly limited. However, the mean pore size may be between 0.005μm to 5.0 μm, 0.01 μm to 2.0 μm, or 0.02 μm to 0.2 μm in consideration of the formation of an ion exchange membrane that is thin and has excellent strength and low electric resistance. The above-mentioned mean pore size as used herein may be a mean flow pore size measured in conformance with the bubble point method (JIS-K3832-1990). Similarly, the porosity of the porous film may be between 20% to 95%, 30% to 90%, or 30% to 60%. In consideration of the thickness of an ion exchange membrane to be finally formed, the thickness of the porous film may be approximately 5 μm to 140 μm, 10 μm to 130 μm, or 15 μm to 55 μm. According to some embodiments, an anion exchange membrane or a cation exchange membrane formed of such porous film may have the same thickness as that of the porous film or up to about 20 μm larger than the thickness of the porous film.

Furthermore, the drug solution holding portion may include a thin film that holds a drug or the like by being impregnated with the drug or the like. Such thin film may have a sufficient ability to hold a drug or the like by being impregnated with the drug or the like, and a sufficient ability (e.g., ion transferability, ion conductivity, etc.) to transfer an ionized drug, which is impregnated thereinto and held by the thin film, to the skin side of an organism under predetermined electric field conditions. Examples of a material that brings together good property of holding a drug by being impregnated with the drug and good ion conductivity include hydrogel forms of acrylic resins (e.g., an acrylic hydrogel film), a segmented polyurethane-based gel film, and an ion-conductive porous sheet to form a gel-like solid electrolyte (e.g., a porous polymer disclosed in JP 11-273452 A using, as a base, an acrylonitrile copolymer comprising at least 50 mol %, between 70 to 98 mol % or more of acrylonitrile and having a porosity of between 20% to 80%). When such drug solution holding portion as described above is impregnated with a drug, an impregnation rate (defined by 100×(W-D)/D (%) where D indicates a dry weight and W indicates a weight after impregnation) may be approximately between 30% to 40%.

Specific examples of an ionic drug according to some embodiments may include anesthetic drugs (e.g., procaine hydrochloride and lidocaine hydrochloride), gastrointestinal disease curing agents (e.g., carnitine chloride), skeleton muscle relaxants (e.g., vancuronium bromide) antibiotics (e.g., a tetracycline-based preparation, a kanamycin-based preparation, and a gentamicin-based preparation), vitamin (e.g., vitamin B2, vitamin B12, vitamin C, and vitamin E), adrenal cortex hormones (e.g., a hydrocortisone-based water-soluble preparation, a dexamethasone-based water-soluble preparation, and a prednisolone-based water-soluble preparation) and antibiotics (e.g., a penicillin-based water-soluble preparation and a chloramphenicole-based water-soluble preparation).

An ionic drug amount may be determined for each individual ionic drug such that a preset effective blood concentration may be obtained for an effective time period upon application of the drug to a patient. The ionic drug amount may be set by one skilled in the art in accordance with, for example, the size and thickness of a drug solution holding portion or the like, the area of a drug release surface, a voltage in an electrode device, and an administration time.

The following conditions may, for example, be adopted as energizing conditions in an iontophoresis device using the electrode assembly according to some embodiments.

(1) Constant current condition such as, for example, 0.1 to 0.5 mA/cm² or 0.1 to 0.3 mA/cm².

(2) Safe voltage condition that realizes the above constant current such as, for example, 50 V or less or 30 V or less.

International Application No. WO 03/037425 A1, described above, describes details about the respective components and operating conditions of such electrode assembly for iontophoresis as described above, and the contents described in the document are also included in various embodiments of the present invention.

In addition, as described above, the electrode assembly for iontophoresis may be used to produce an iontophoresis device. Therefore, according to another embodiment of the present invention, the electrode assembly may be used for iontophoresis in the production of an iontophoresis device. According to another embodiment of the present invention, a kit may be capable to produce an iontophoresis device including at least the electrode assembly for iontophoresis according to various embodiments of the present invention.

The various embodiments described above can be combined to provide further embodiments. All of the U.S. patents, U.S. patent application publications, U.S. patent applications referred to in this specification and/or listed in the Application Data Sheet, are incorporated herein by reference, in their entirety. Embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An electrode assembly for iontophoresis, characterized by comprising:
an electrode connected to an electric power source having the same polarity as that of a drug component of an ionic drug;
an electrolyte solution holding portion holding an electrolyte solution by being impregnated with the electrolyte solution, the electrolyte solution holding portion being placed adjacent to the electrode;
an ion exchange membrane selecting an ion having a polarity opposite to that of a charged ion of the ionic drug, the ion exchange membrane being placed adjacent to the electrolyte solution holding portion;
a drug solution holding portion holding the ionic drug by being impregnated with the ionic drug, the drug solution holding portion being placed adjacent to the ion exchange membrane; and
an ion exchange membrane selecting an ion having the same polarity as that of the charged ion of the ionic drug, the ion exchange membrane being placed adjacent to the drug solution holding portion,
the electrode assembly being frozen.

2. A kit for producing an iontophoresis device, characterized by comprising at least the electrode assembly for iontophoresis according to claim 1.

3. A use of the electrode assembly for iontophoresis according to claim 1 in production of an iontophoresis device.
